(19) United States
(12) Patent Application Publication
(10) Pub. No.: US 2005/0098465 A1
(43) Pub. Date: May 12, 2005

(54) Packaged scented toilet paper holder

(76) Inventor: Thomas Wolf, Highland Park, IL (US)

Correspondence Address: James B. Conte;Barnes & Thornburg, P.O. Box 2786, Chicago, IL, 60690-2786, US

(21) Appl. No.: 10/706,628

(22) Filed: Nov. 12, 2003

(51) Int. Cl. B65D 85/00; B65H 75/18; A61L 9/04

(52) U.S. Cl. 206/410; 239/52; 428/905; 242/599; 242/905

(57) ABSTRACT

A scented toilet paper roller is sealed and encompassed in a metalized wrapper. The scented toilet paper roller includes a first roller half and a second roller half. The first and second roller halves are telescopically joined and form a hollow roller chamber. A compression spring is disposed within the hollow roller chamber. Scented beads are disposed within the hollow roller chamber. Scented oil is disposed on the scented beads. The roller halves have a plurality of vents therein. The roller halves can be thermoplastic.

### FIELD OF INVENTION

The present invention concerns a wrapped scented toilet paper roller.

### BACKGROUND OF THE INVENTION

There are many products on the market aimed at improving the scent of the air in household rooms. One such product is a scented toilet paper roller. A known roller has a cavity containing scented beads and a number of openings to allow the scent from the beads to be released into the air. The roller supports a roll of toilet paper in a conventional toilet paper holder.

An example of a known scented roller is described in U.S. Pat. No. 4,759,510, Singer. The patent is directed towards a universal scent-emitting toilet paper roller. The roller is described as including three components molded of thermoplastic material. Two of these components are identical vented hollow closed ended generally cylindrical roller halves, each half containing tongues and slots which permit the two roller halves to be axially slidably joined together. The third thermoplastic component is a helical coiled spring designed to fit inside the joined together roller halves and urge the two halves into their maximum axially extended position. A recessed truncated conical projection axially centered on the closed end of each roller half fits the assembled roller into a rolled paper dispenser. A quantity of scent-emitting pellets are contained within the hollow interior of the joined together roller halves to be tumbled about as the roller is rotated by the removal of paper, thus causing scent to be emitted into the atmosphere through vents in the roller.

### SUMMARY OF THE INVENTION

One embodiment of the present invention is an individually-wrapped, scented toilet-paper roller. The roller is enclosed in a metalized film wrapper. The wrapper may be metalized polyester (PET), as this is preferred.

### BRIEF DESCRIPTION OF THE DRAWINGS

FIG. 1 is a side perspective view of an example of a wrapped scented toilet paper roller which embodies the features of the present invention; the wrapping is partially cut away.

FIG. 2 is a cross-sectional view of the wrapped scented toilet paper roller shown in FIG. 1 and taken along view lines 2.

FIG. 3 is a blown-up sectional view of the wrapping shown in FIGS. 1 and 2.

FIG. 4 is a side perspective view of the scented toilet paper roller shown in FIGS. 1 and 2 without any wrapping.

FIG. 5 is a left side view of the roller shown in FIG. 4.

FIG. 6 is a right sided view of the scented toilet paper roller shown in FIG. 4.

FIG. 7 is an exploded view of the scented toilet paper roller shown in FIG. 4.

FIG. 8 is a sectional view of the scented toilet paper roller shown in FIG. 4 with the section taken along the roller's longitudinal axis.

FIG. 9 is a close-up view of one of the scented beads shown in FIG. 7 wherein the scented bead has a layer of scented oil thereon.

FIG. 10 is a close-up partial sectional view of the bead layered with oil shown in FIG. 9.

FIG. 11 is a side perspective view of the scented roller in the contracted position.

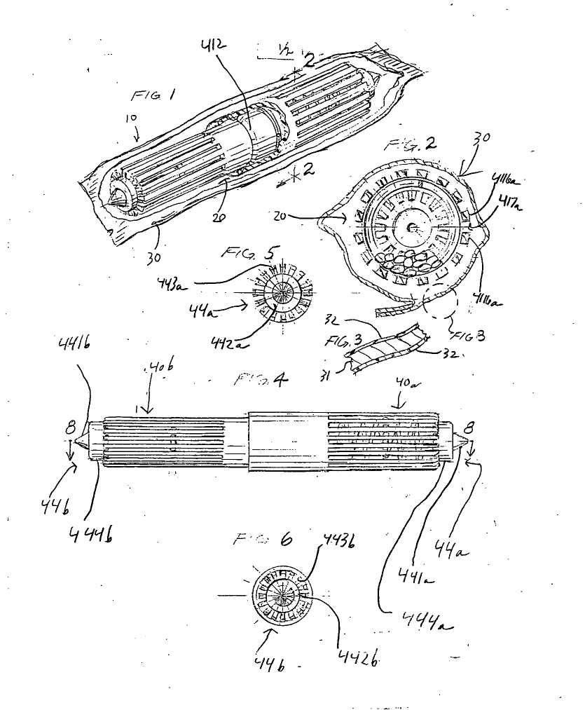

### DETAILED DESCRIPTION OF THE EMBODIMENT

One example of the invention is further described in detail as follows. The wrapped scented toilet paper roller 10 includes a scented toilet paper roller 20 sealed and encompassed in a metalized wrapper 30. The scented toilet paper roller 20 comprises a roller 40a, 40b, a compression spring 50 disposed within the roller, scented beads 60, and scented oil 70 disposed on the scented beads.

The roller 40a, 40b includes a first roller half 40a and a second roller half 40b. The first and second roller halves 40a, 40b each have a curvilinear wall 41a, 41b disposed around the rollers longitudinal axis 90. Each curvilinear wall 41a, 41b extends along the length of the longitudinal axis 90. Each curvilinear wall has an inner surface 42a, 42b. Each inner surface defines at least in part, hollow roller chambers 43a, 43b.

Each roller half 40a, 40b has an end wall 44a, 44b. Each end wall 44a, 44b is disposed towards an end of each roller half 40a, 40b. Each roller half, opposite each end wall 44a, 44b, has an open end 45a, 45b, Each open end opens into each roller halves respective roller chamber 43a, 43b.

The first roller half 40a has an outer diameter 400a greater than the outer diameter 400b of the second roller half 40b. The second roller half open end 45b is disposed within the first roller half open end 45a so that the first roller chamber 43a and second roller chamber 43b form a continuous roller chamber 43a, 43b extending along the length of the rollers longitudinal axis 90.

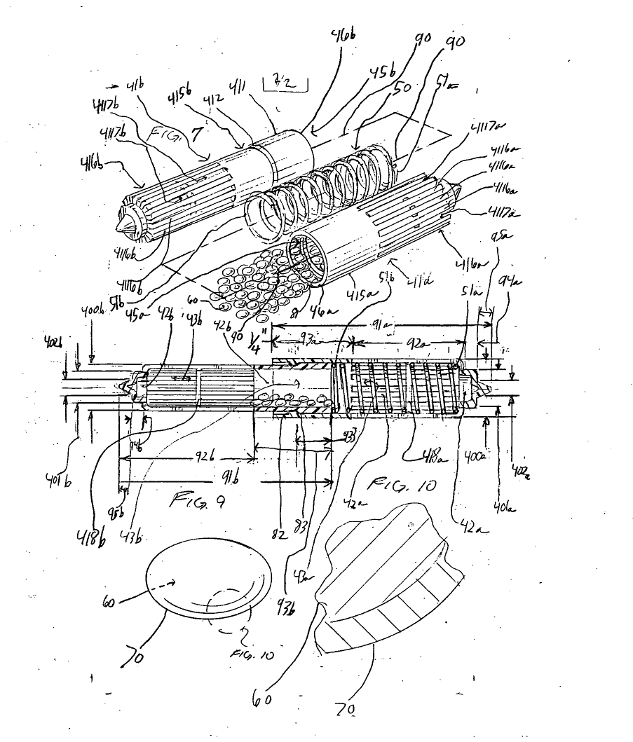

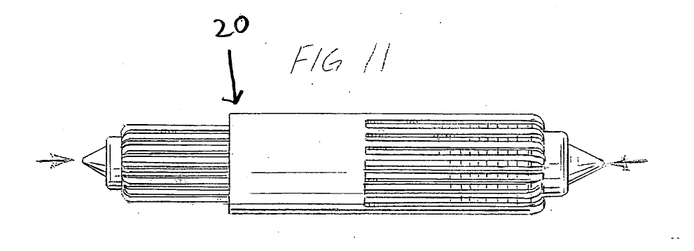

The second roller half 40b is telescopically engaged within the first roller half 40a. Thus the first and second roller halves are slidably movable, relative to each other, along the rollers longitudinal axis 90 to allow the roller 40a, 40b to be adjusted from a retracted position. See FIG. 11 to an expanded toilet tissue installation position. See FIG. 4.

The compression or coil spring 50 is disposed within the roller chamber 43a, 43b. The compression or coil spring has a first end coil 51a abutting up against first roller half end wall 44a. The compression spring 50 has a second end coil 51b axially opposite the first end coil 51a. The second end coil 51b abuts up against an end wall 46b bounding the open end 45b of the second roller half 40b. An end wall 46a also bounds open end 45a of the first roller half 40a. The end wall 46a forms an outermost shoulder of the scented roller 20.

In the expanded installation position, the compression spring 50 biases the roller halves with a force so each roller half is pushed in a direction axially opposite the other roller half. The roller 40a, 40b has a retention mechanism to ensure that the first roller half 40a and second roller half 40b, in the expanded installation position, remain telescopically engaged in an orientation which applies a compression force to the spring. A force applied to both or either roller half 40a, 40b, in a direction toward a roller half open end 45a, 45b and along the roller's longitudinal axis 90 will cause the roller halves to further compress spring 50 and to each move toward the other into a contracted position. In the present embodiment, the compression spring is disposed within the first roller chamber 43a. Other dispositions of the roller spring are possible.

In more detail, the retention mechanism includes a protrusion 81. The protrusion 81 extends inward towards the longitudinal axis 90 from the first roller half inner surface 42a. The protrusion 81 extends in a direction transverse to longitudinal axis 90. Although the protrusion is shown as an unbroken cylindrical shoulder, it could have other configurations. The protrusion has an end surface 82 facing the first roller half open end 45a. The end surface 82 is oriented in the same direction relative to the longitudinal axis as is the first roller half open-end wall 46a. The protrusion 81 has a ramped surface 83. The ramped surface 83 slopes inward from end surface 82 towards the first roller half inner surface 42a.

The retention mechanism further includes a portion 411 of the second roller half wall 41a. The portion 411 has an expanded diameter. The surface of wall portion 411 having the expanded diameter is brushed as opposed to slick. A ledge 412 is formed between wall portion 411 and an adjacent remaining portion of the second roller half wall 41a. In the roller expanded position, the expanded wall portion 411 is axially further away from first roller half open end 45a than is the protrusion 81. The spring remains slightly compressed, thereby exerting forces on each roller half 40a, 40b to force the ledge 412 to abut up against the protrusion 81.

Each end wall 44a, 44b of each roller half 40a, 40b has distinct portions. Each end wall has a conical protrusion 441a, 441b tapering along longitudinal access 90 in a direction opposite its respective roller halves open end. Each end wall also includes a second plateau 442a, 442b perpendicular to the longitudinal axis. Each conical portion 441a, 441b extends outwardly from its respective second plateau 442a, 442b, along longitudinal axis 90, and away from the open end of its respective roller half. Each end wall further includes a first plateau 443a, 443b concentric to and parallel to the second plateau. Each first plateau 443a, 443b is joined to its respective second plateau 442a, 442b and spaced axially therefrom by a cylindrical shoulder 444a, 444b. Each cylindrical shoulder 444a, 444b is perpendicular to the first and second plateau which it joins. Each first plateau 443a, 443b is comprised of a series of ribs or spokes disposed circumferentially around its respective cylindrical shoulder. The first plateau 443a, 443b can be referred to as a first platforms. The second plateaus 442a, 442b can be referred to as a second platforms. The second plateaus protrude from the their respective first plateaus in the axial direction and away from the open end of their respective roller halves.

The roller walls 41a, 41b each have solid cylindrical portions 415a, 415b and ribbed portions 416a, 416b. Protrusion 81 emanates from the inward surface 42a of the first roller half solid wall portion 415a. The extended wall 411 portion forms part of the second roller half solid wall 415b. The ribbed sections 416a, 416b of each roller half includes a plurality of ribs 4116a, 4116b which are substantially parallel to each other. The ribs are also substantially parallel to the longitudinal axis 90. The ribs are disposed about the longitudinal axis 90. Sixteen of the ribs 4116a form the ribbed portion of the first roller half. Sixteen of the ribs 4116b also form the ribbed portion of the second roller half. The ribs 4116a on the first roller half are spaced such that a chasm 417a of about 1/16 of an inch exists between each pair of adjacent ribs. A space between each of the ribs on the second roller half forms a chasm 417b between each rib which is also 1/16 of an inch. The ribs 4116b of the second roller half have a thickness slightly less than the thickness of the ribs 4116a of the first roller half. The second roller half has an outer diameter 400b of about ¾ of an inch. The second roller half ribs have a width to ensure the 1/16th of an inch spacing between each rib. The first roller half has an outer diameter 400a which is about 15/16 of an inch. The ribs of the first roller half have a width to allow for the 1/16 of an inch spacing between each rib.

Each ribbed section 416a, 416b of each roller half has an internal support 418a, 418b to help stabilize the relative position of each rib relative to the roller's longitudinal axis 90. The support 418a for the ribbed section of the first roller half forms an internal hoop, is transverse to the roller's longitudinal axis, and parallel to the first roller half end wall 44a. The hoop extends along the internal surface 42a of the ribs 4116a forming the first roller half ribbed portion 416a. The support for the ribs 4116b of the second roller half is similarly fashioned as the support for the first roller half.

The first roller and second roller half each have a total length 91a, 91b of 3⅜″. The ribs of the first roller half have a length 92a of 1¾″. The ribs of the second roller half also have a length 92b of 1¾″. The wall portion having the expanded diameter 411 has a length 933 of 9/16 of an inch. The first and second roller halves solid wall portions each have a longitudinal length 93a, 93b of 1 3/16″.

The second plateau on each end wall is an axial distance 94a, 94b from its respective first platform of 3/16 of an inch. The conical portion of each end wall has a length 95a, 95b which extends about 4/16 of an inch axially outward from the second plateau. The second platform of the second roller half has a diameter 401b of about ½″. The second platform of the first roller half has a diameter 401a of about ⅝″. The base portion of each end wall's conical member has a diameter 402a, 402b of about ¼″.

The proportional relationship between the dimensions of each part of the roller are believed to have importance. Proportional relationships, though not specifically stated herein are easily calculated based on the dimensional disclosures herein.

The scented roller is sealed within metalized wrapper 30. The wrapper is disposed around the scented roller 20 so as to provide a substantially airtight barrier around the roller. The metalized wrapper is preferably aluminum 31 coated with polyester 32. The beads disposed within the roller are scented. The beads are made out of a resilient soft plastic. The beads 60 are scented. The scented beads have applied thereto a layer of scented oil 70.

1. A wrapped scented toilet paper roller comprising:
a first roller half;
a second roller half, said first and second roller halves adjustably engaged with each other so that each is slidably movable relative to the other along a longitudinal axis of said scented toilet paper roller;
a curvilinear wall forming a portion of said first roller half;
a curvilinear wall forming a portion of said second roller half;
a solid cylindrical portion and a ribbed portion adjacent to each other and each forming at least a portion of said curvilinear wall of said first roller half;
a solid cylindrical portion and a ribbed portion adjacent to each other and forming at least a portion of the second roller half curvilinear wall;
a plurality of sixteen ribs spaced such that a chasm of about 1/16″ exists between adjacent ribs of said plurality of sixteen ribs, said plurality of sixteen ribs forming said rib portion of said first roller half;
a plurality of sixteen ribs spaced such that a chasm of about 1/16″ exists between adjacent ribs of said plurality of sixteen ribs, said plurality of sixteen ribs forming said rib portion of said second roller half;
a plurality of scented beads disposed within a hollow chamber formed by said first and second roller halves;
an amount of scented oil disposed over at least some of said plurality of scented beads; and
wherein said first roller half has an outer diameter of about 15/16″; said second roller half has an outer diameter of about ¾″; and
wherein said solid cylindrical portion of said second roller half includes a portion with an expanded diameter such that this portion has an outer diameter greater than ¾″.

2. The wrapped scented toilet paper roller of claim 1 wherein said wrapping comprises a layer of aluminum at least partially coated with polyester; said metalized wrapper providing an airtight barrier around said scented toilet tissue roller.

3. The wrapped scented toilet paper roller of claim 1 wherein the roller halves are thermoplastic.

4. The wrapped scented toilet paper roller of claim 1 further comprising:
a cylindrical support bracing the sixteen ribs on the second roller half;
a cylindrical support bracing the sixteen ribs on the first roller half.
